(19) United States
(12) Patent Application Publication
(10) Pub. No.: US 2004/0147950 A1
(43) Pub. Date: Jul. 29, 2004

(54) Atraumatic dilator for human mammary duct

(76) Inventors: Richard L. Mueller JR., Jackson, WY (US); James Kermode, Los Altos, CA (US); David Gallup, Alameda, CA (US)

Correspondence Address: OLSON & HIERL, LTD., 36th Floor, 20 North Wacker Drive, Chicago, IL, 60606, US

(21) Appl. No.: 10/350,631

(22) Filed: Jan. 24, 2003

(51) Int. Cl. A61M 29/00

(52) U.S. Cl. 606/191

(57) ABSTRACT

An atraumatic dilator suitable for the dilation of mammary ducts is a filament with opposed end portions. At least one end portion is pliable and has a taper that terminates in a rounded tip.

### TECHNICAL FIELD OF THE INVENTION

The invention relates to a device for dilating a mammary duct in conjunction with a duct cannulation procedure. Specifically, the invention relates to a device that is at least partially pliable, having at least one tapered portion for dilating a mammary duct.

### BACKGROUND OF THE INVENTION

Breast cancer is one of the health threats most feared by women, and is the most common form of cancer in women. A key to treatment is early detection. For example, an annual mammogram is a method that has been used in hopes of early detection of breast cancer. One problem with mammography is that such an imaging technique can only find breast cancer once it has taken form. All too often, breast cancer is discovered at a stage that is too far advanced, when therapeutic options and survival rates are severely limited. As such, more sensitive and reliable methods and devices are needed to detect cancerous, pre-cancerous, and other cancer markers of the breast at an early stage. Such methods and devices could significantly improve breast cancer survival. While breast cancer is most common among women, in rare instances the human male may also have occurrences of breast cancer.

Other methods of detecting breast cancer are based on the fact that a vast majority of instances of breast cancer begins in the lining of mammary ducts. Studies have shown that fluid within the mammary duct contains high levels of breast cancer markers, and that an estimated 80%-90% of all breast cancers occur within the intraductal epithelium of the mammary glands. Fluid within the breast ducts contains an assemblage and concentration of hormones, growth factors and other potential markers comparable to those secreted by, or acting upon, the surrounding cells of the alveolar-ductal system. Likewise, mammary fluid typically contains cells and cellular debris, or products that can also be used in cytological or immunological assays.

One such method of obtaining samples of cells from a mammary duct is ductal lavage. This method comprises the rinsing of a mammary duct with a saline solution or the like and collection of the solution along with any cells and cellular debris from the mammary duct. Typically, a catheter having an internal lumen is used to introduce the solution into the mammary duct. Conventional catheters include distal portions that may be introduced into a mammary duct and advanced using an internally positioned dilator. The use of a dilator is preferred inasmuch as a catheter has an open end. Mammary ducts terminate at the surface of a human nipple at an orifice and are closed by a sphincter muscle. This sphincter muscle must be passed in order introduce a solution such as a saline solution for ductal lavage. A catheter's open end, however, usually terminates at sharp angles or narrow edges that can damage a mammary duct as well as the sphincter muscle. A dilator, which may pass through the lumen of a catheter, does not have an open end, however.

Conventional dilators are made of a substantially rigid metal or hard plastic. In use, the dilator is extended through the distal end of a catheter past the ductal sphincter muscle. The dilated sphincter is then more easily penetrated by the catheter. Also, since the dilator is sized only slightly smaller than the catheter lumen, the sharp distal end of the catheter is less likely to damage the mammary duct or the sphincter muscle. The dilator can then be removed in order to flush the mammary duct with a lavage solution.

Mammary duct damage, however, may still occur when a dilator is utilized. In particular, rigid dilators, due to their rigid nature, are capable of causing damage to a mammary duct and its sphincter. Use of softer pliable materials to dilate mammary ducts have been tried. For example, use of a polypropylene monofilament guiding suture with a catheter has been attempted, but with limited success. Accordingly, there continues to exist a need for an improved atraumatic mammary duct dilator.

### SUMMARY OF THE INVENTION

An atraumatic dilator for guiding a catheter past a mammary duct sphincter muscle and into a mammary duct is provided. The atraumatic dilator comprises a filament having a proximal end portion and a distal end portion. At least one end portion of the filament is of a pliable material. The end portions of the atraumatic dilator can be tapered and also can have a rounded tip. The distal and proximal end portions of the dilator can be used interchangeably.

The atraumatic dilator may be pliable over its entire length if desired. For example, the filament may be made of polypropylene, polyurethane or other pliant plastic material. Accessing the mammary duct with such a soft, pliable probe having a rounded tip is not likely to cause damage to the mammary duct, mammary duct orifice or sphincter muscle. Most mammary ducts may be initially accessed with at least the pliable distal end portion of the atraumatic dilator embodying the present invention.

In some instances, however, a pliable probe will be insufficient to dilate the mammary duct. In such situations, the present, atraumatic dilator facilitates a follow-up procedure with a rigid probe. An alternative embodiment of the present invention provides a single atraumatic dilator that is pliable over one end portion, while rigid over another, opposite end portion. For example, about one-half of the filament may be made of polypropylene or other pliable material, while the other one-half is a filament of stainless steel. If a patient's mammary duct is inaccessible with the pliable portion of the filamentary length, the physician or medical personnel may turn the atraumatic dilator around and use the more rigid portion thereof to access the mammary duct. Alternatively, introduction of the pliable portion of the dilator can be followed by introduction of the more rigid portion for further dilation. A stiffener may also be incorporated with the dilator to provide sufficient resistance to bending while still presenting a relatively soft atraumatic probe.

The entire dilator may be comprised of a polypropylene, polyurethane or like material having different levels of flexural rigidity at different portions of the dilator. For example, one end portion of the dilator may have a relatively lower flexural rigidity than the other end portion of the dilator. Different portions of the dilator can be color coded, if desired, so that different colors can indicate to the physician or medical personnel a different degree of pliability or flexural rigidity.

The atraumatic dilator of the present invention also has a tapered end portion that terminates in a rounded tip. Such a tapered end portion enables the mammary duct orifice, the sphincter muscle and the mammary duct to be more easily penetrated by the dilator. The rounded tip also lessens the possibility of damage because there are no edges to cause harm to the mammary duct orifice, the sphincter muscle or the mammary duct. The atraumatic dilator also preferably includes a tactile response region to provide the physician with tactile feedback on the insertion of the dilator. All or a portion of the atraumatic dilator may also be coated with a friction reducing coating, e.g., polyfluorocarbon coating, to further aid in the dilation process.

The rounded tip may be made of the same material as the distal end portion of the dilator, or may be made of a different material. The rounded tip may be comprised of a relatively softer material to further lessen the possibility of duct injury during use.

The end portions of the present dilator preferably include a taper, more preferably a taper having an angle with the longitudinal center line of the dilator (draft) in the range of about 2 degrees to about 15 degrees, and most preferably in the range of about 2 degrees to about 4 degrees. That is, the included angle of the taper more preferably is about 4 to about 30 degrees, most preferably about 4 to about 8 degrees. The taper may also include a threaded region to assist in insertion of the dilator. The dilator can also include a depth marker, if desired.

### BRIEF DESCRIPTION OF THE DRAWINGS

In the drawings,

FIG. 1 is a side view of a preferred embodiment of an atraumatic dilator embodying the present invention;

FIG. 2 is an enlarged, fragmentary schematic view of an atraumatic dilator about to be inserted into a mammary duct;

FIG. 3 is an enlarged fragmentary view of a tapered end portion of the atraumatic dilator shown in FIG. 1;

FIG. 4 is an enlarged fragmentary side view of the opposite, untapered end portion of the atraumatic dilator shown in FIG. 1;

FIG. 5 is an enlarged fragmentary side view of an alternate embodiment of a tapered end portion of an atraumatic dilator;

FIG. 6 is a side view of a pliable atraumatic dilator embodying the present invention;

FIG. 7 is an enlarged side view of the dilator of FIG. 6 and including an optical fiber core;

FIG. 8 is a side view of yet another atraumatic dilator embodying the present invention;

FIG. 9 is an enlarged side view of an atraumatic dilator embodying the present invention and including an internal stiffening core;

FIG. 10 is an enlarged side view partly in section of an atraumatic dilator embodying the present invention and including an external stiffening sheath;

FIG. 11 is a perspective view of an atraumatic dilator embodying the present invention and including an external stiffening clamp;

FIG. 12 is an enlarged cross-sectional side view of another stiffening clamp and provided with an elongated slot;

FIG. 13 is a perspective view of an embodiment of a retaining device for use with an atraumatic dilator embodying the present invention;

FIG. 14 is an enlarged fragmentary side view of an end of the atraumatic dilator provided with a tactile response region;

FIG. 15 is an enlarged fragmentary side view of an end of the atraumatic dilator provided with an alternative tactile response region; and

FIG. 16 is an enlarged fragmentary side view of a rounded, self-threading of the atraumatic dilator embodying the present invention.

### DESCRIPTION OF THE PREFERRED EMBODIMENT OF THE INVENTION

The invention disclosed herein is, of course, susceptible of being embodied in many different devices. Shown in the drawings and described herein below in detail are preferred embodiments of the invention. It is to be understood, however, that the present disclosure is an exemplification of the principles of the invention and does not limit the invention to the illustrated embodiments.

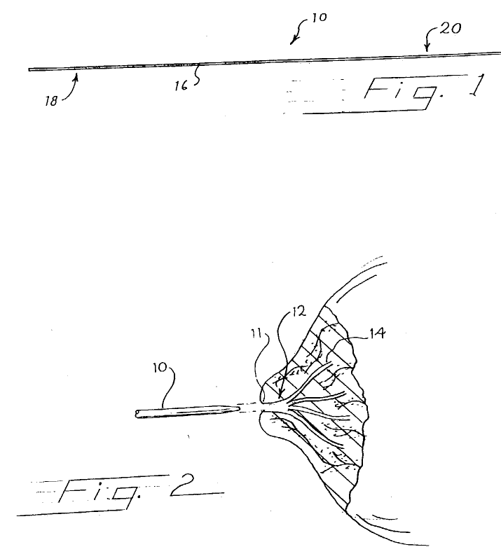

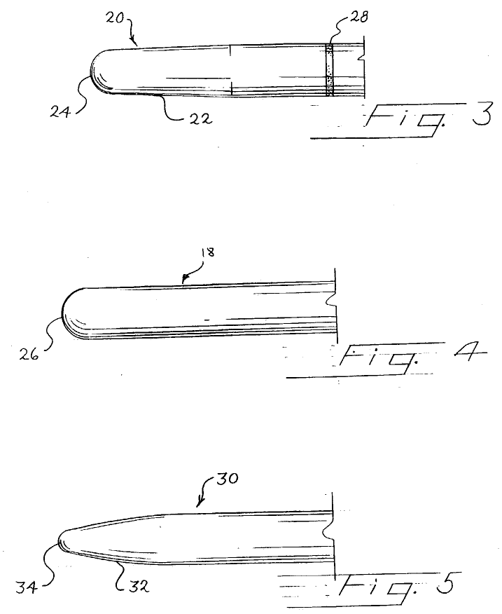

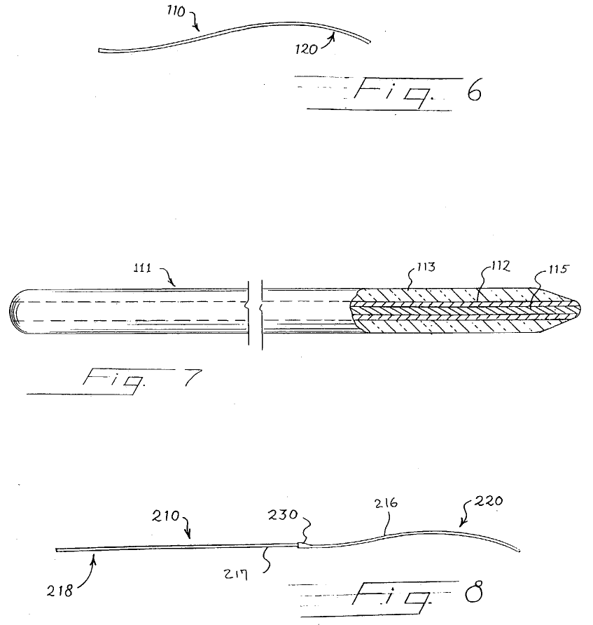

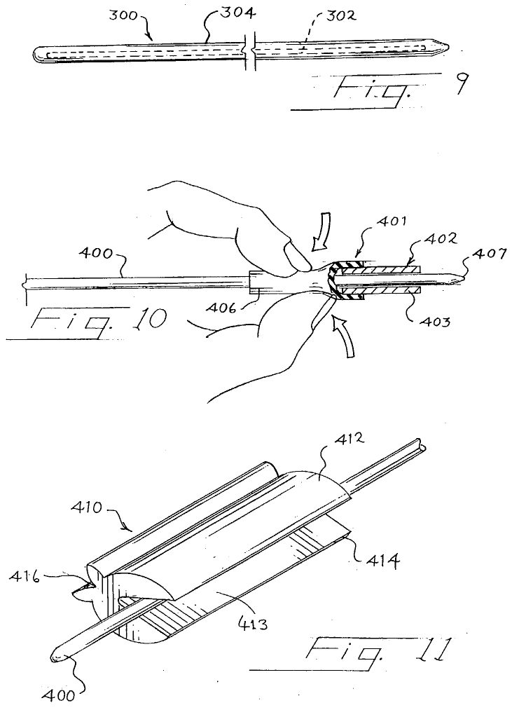

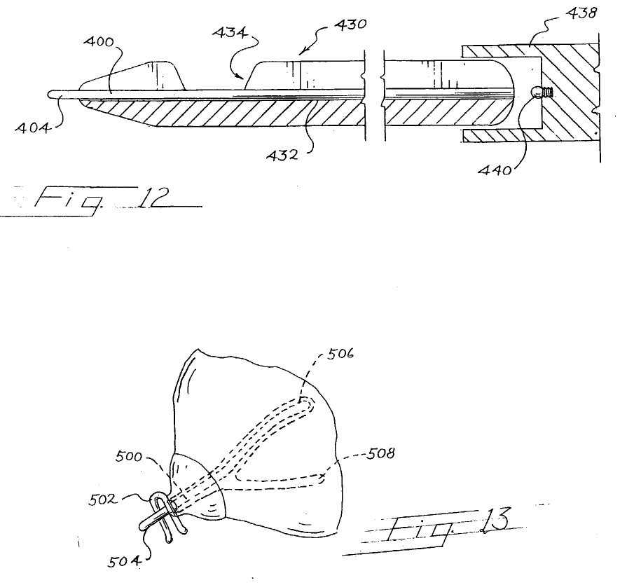

Referring to FIGS. 1 and 2, an atraumatic dilator 10 for guiding a catheter (not shown) through a mammary duct orifice 11, past a mammary sphincter muscle 12 and into a mammary duct 14 is provided. The atraumatic dilator 10 comprises a filament 16 having a rounded end portion 18 and a rounded end portion 20. At least one of the rounded end portions is tapered. At least a portion of the filament 16 is pliable. Depending on use, either end portion 18 or end portion 20 can be a distal end portion or a proximal end portion.

As shown in FIG. 3, the end portion 20 of the atraumatic dilator has a taper 22 extending outwardly to a rounded tip 24 at an included angle of about 4 degrees (about 2° draft). The taper 22 of the distal end portion 20 of the dilator 16 is preferably tapered about 2 degrees to about 15 degrees, and more preferably about 2 degrees to about 4 degrees. The end portions 18 and 20 can have the same degree of taper, or the degree of taper may differ, as desired. The taper extends inwardly, toward the body of the dilator, preferably for about 0.035 inches (about 0.9 millimeters). A penetration depth marker 28 may also be included on the atraumatic dilator 10. It is preferred that the penetration depth marker 28 be located approximately 1 centimeter from the rounded tip 24. In embodiments wherein the proximal portion of the dilator also includes a taper and rounded tip, another depth marker may be similarly located. End portion 18 of the atraumatic dilator, opposite end portion 20, may have a square end (not shown), or a rounded tip 26 as shown in FIG. 4 if end portions 18 and 22 are to be interchangeable in use. Preferably, the end portion 18 has a taper similar to taper 22 of end portion 20 as shown in FIG. 3. The taper 22 permits the mammary duct orifice 11, the mammary sphincter muscle 12 and the mammary duct 14 to be more easily penetrated by the dilator 10. The rounded tip, such as tips 24 and 26 also lessen the possibility of damage because there are no edges to cause abrasions or cuts. The atraumatic dilator 10 may also be coated with a friction minimizing coating, e.g., a polyfluorohydrocarbon coating, to minimize friction and further aid in the penetration and dilation procedure.

The rounded tip 24 may be of the same material as the distal end portion 20 of the dilator 16. In another embodiment, the rounded tip may be of a different material, preferably a softer material, to further lessen the possibility of trauma.

FIG. 5 illustrates a dilator 30 with a taper 32 having an included angle of about 30 degrees (about 15° draft) and rounded tip 34.

FIG. 6 illustrates an embodiment of the present invention where the atraumatic dilator 110 is pliable over its entire length. The distal portion 120 of the atraumatic dilator 110 preferably has a taper and a rounded tip similar to that shown in either FIG. 3 or 5. The atraumatic dilator 110 may be spray or dip coated with an anti-friction agent such as medical grade silicone, e.g., a MDX silicone elastomer available from Dow Corning Corporation, Midland, Mich., if desired.

In certain situations, it is desirable for the dilator to have light transmitting properties, such as those of optical fibers. For example, dilator 111 may be comprised of a core material 115 and a cladding material 112 as shown in FIG. 7. The relative indices of refraction of core material 115 and cladding material 112 are such that light entering one end of dilator 111 travels the full length of dilator 111. A polymer coating 113 having the desired durometer may also be included. If it is desired that light be permitted to escape along some or all of the length of dilator 111, the cladding material 112 may define voids or apertures which permit light to escape.

In yet another embodiment, illustrated in FIG. 8, the atraumatic dilator 210 may be pliable over an end portion 220, while rigid over end portion 218. For example, the end portion 220 of the monofilament 216 may be made of polypropylene, polyurethane or other pliable material, whereas the end portion 218 of wire 217 is made of stainless steel. If a patient's mammary duct is inaccessible with the pliable end portion 220, the physician or medical personnel may turn the atraumatic dilator 210 around and use the rigid end portion 218 to access the mammary duct initially. The monofilament 21 may be grafted to sire 217 at joint 230. Alternatively, a crimp or bond joint may provide the joint. The rigid portion and the pliable portion of the dilator can also be joined together with a coupling sleeve, or the like.

In a preferred embodiment of the present invention, integrally formed distal and proximal end portions utilize two polymers of different durometer values that are insert molded or cast to form a constant diameter joint. A double ended dilator with different levels of rigidity for the opposite ends thereof is molded or cast in a suitable mold that defines a cavity with a tapered section and a rounded section. The mold is filled sequentially or simultaneously with compatible polymers during the molding process. A suitable polymer is polyurethane which is available in a wide spectrum of durometer values for molding and casting grades. As discussed above, an optical fiber may also be included as a core for the dilator.

In certain situations, a polymer having a lower rigidity may better dilate a nipple orifice with the use of a stiffener. Where a polymer is too soft, it will not have the lateral strength to resist merely being bent as one attempts to insert the dilator past the sphincter. One example of such a stiffener is shown in FIG. 9. In this embodiment, an internal stiffening core 302 e.g., a wire or an optical fiber, is incorporated into dilator 300 along with the polymeric material during the molding process. In this manner, enhanced overall dilating stiffiess is achieved while maintaining surface softness for the dilator 300. Depending on the durometer of the polymer that constitutes outer polymeric sheath 304, the stiffening core 302 may extend the entire length of the dilator 300, or only along that portion of the dilator 302 needing lateral support. The stiffening core 302 may be comprised of a light transmitting material, such as an optical fiber, to transmit light from one end of dilator 302 to the other.

An alternative stiffener is disclosed in FIG. 10. Dilator 400 is received within external sheath 401 that has a rigid portion 402 provided by sleeve 403 and flexible portion 406. Distal end 404 with a rounded tip 407 protrudes from the rigid portion 402. Rigid portion 402 has an inner diameter which is slightly larger than the outer diameter of dilator 400. It is preferred that the inner diameter be in the range of about 0.002 inches to about 0.010 inches larger (about 0.05 mm to about 0.25 mm) than the outer diameter of dilator 400. Coupled to or integral with the rigid portion is flexible portion 406. Flexible portion 406 is manually squeezable. The physician, by pinching flexible portion 406, secures dilator 400 within the external sheath 401 such that the distal end 404 of the dilator 400 is more easily inserted into a mammary duct.

Another embodiment of a stiffener is shown in FIG. 11. The external stiffening clamp 410 is configured with a pair of longitudinally extending lobes 412 and 414 that define a V-shaped trough 413 therebetween and are connected by a longitudinally extending hinge portion 416. Dilator 400 is immobilized between lobes 412 and 414 when the external stiffening clamp 410 is pinched. Longitudinally extending hinge portion 416 preferably is resilient so that release of manual pressure on lobes 412 and 414 results in release of the dilator 400. Lobes 412 and 414 can be molded of an elastomeric material unitary with hinge portion 416.

Yet another alternative stiffener is shown in FIG. 12. In this particular embodiment, stiffener 430 defines a longitudinal trough 432, which is adapted to receive a dilator 400 therein. A recess 434 is also provided whereby the practitioner can press down on dilator 400. Distal end portion 404 of dilator 400 protrudes out of distal end 438 of stiffener 430. In situations where the dilator 400 includes an optical fiber, a light cable adaptor 438 with a light source 440 is used to receive stiffener 430, and provide light through dilator 400 to illuminate a target area on the nipple. The stiffener 430 is of a length such that the dilator 400 is fully received therein, except for protruding end portion 404.

In some circumstances, e.g., in the case that a patient must return for further examination after a biopsy is taken, it may be desirable to leave the dilator in the mammary duct. To that end, as shown in FIG. 13, a dilator 500 may also include a low profile retaining device such as collar 502, which is affixed to an approximately 2 mm of an exposed end portion 504 of the dilator 500. The collar 502, preferably made of a bio-compatible material, prevents the end portion 504 of the dilator 500 from slipping into the mammary duct 508 during the time between patient visits. The dilator 500 acts as a guide for an introducer or surgical cut-down (not shown) such that the correct branch 506 of the mammary duct 508 terminating at the nipple orifice 510 is easily located during the later procedure. Any excess length of the dilator extending beyond the collar 502 may be severed such that the clamp 502 and dilator 500 are as unobtrusive as possible when in place. Alternatively, where the dilator 500 is of a material that is sufficiently pliable to be knotted upon itself, such as with surgical suture type material, a knot may also serve as a retaining device and can be used to keep the dilator from being inadvertently lost inside a mammary duct. A flexible, internally barbed washer can also serve as a retaining device.

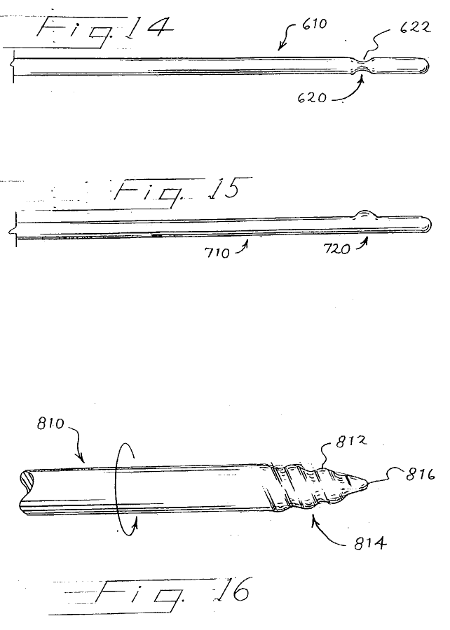

Penetration of the sphincter muscle with an atraumatic dilator is a blind procedure in that the physician cannot actually see when the dilator has passed the sphincter muscle. To that end, a tactile signaling element can be included as a feature of the dilator. Shown in FIG. 14 is one embodiment of such a tactile signaling element. Dilator 610 includes a recessed region 622, on its distal end 620. As the recessed region 622 of dilator 610 travels past the sphincter muscle, a change in resistance to the dilator will be felt by the physician. Alternatively, as shown in FIG. 15, a raised region 722, such as a bump or a ridge, on the distal end portion 720 of dilator 710 can provide tactile feedback as well.

If desired, the taper on the distal end of a device embodying the present invention may include threading to further aid in advancing the end of the dilator past the sphincter muscle. FIG. 16 shows dilator 810 provided with a self-threading end portion that terminates in a rounded tip 816. Threads 812 are provided on the taper 814. As the dilator 810 is rotated about its longitudinal axis, the threads 812 cause the dilator 810 to be drawn into the mammary duct. Preferably, the threads 812 have a rounded shape to lessen the possibility of trauma to the sphincter muscle and the duct.

The flexural rigidity of dilators embodying the present invention can be in the range of about 1 g·cm² to about 100 g·cm². The pliant end portion of the dilator preferably has a flexural rigidity in the range of about 1 g·cm² to about 2 g·cm², a diameter of about 0.010 inches to about 0.015 inches (about 0.25 mm to about 0.4 mm), and a length of about 3 inches to about 12 inches (about 76 mm to about 305 mm). The rounded ends preferably have a radius of about 0.002 inches to about 0.007 inches (about 0.05 mm to about 0.18 mm), depending upon taper and the length of the taper. The more rigid end portion of the dilator preferably has a flexural rigidity of about 90 g·cm² to about 100 g·cm², a diameter of about 0.010 inches to about 0.015 inches (about 0.25 mm to about 0.4 mm), and a length of about 3 inches to about 12 inches (about 76 mm to about 305 mm). The rounded end thereof preferably has a radius of about 0.002 inches to about 0.007 inches (about 0.05 mm to about 0.18 mm), depending upon taper and the length of the taper.

The foregoing description is to be taken as illustrative, but not limiting. Still other variants within the spirit and scope of the present invention will readily present themselves to those skilled in the art.

We claim:

1. An atraumatic dilator suitable for guiding a catheter in a mammary duct dilation procedure and comprising:
a filament having opposed end portions, at least one end portion of the filament being pliable, having a taper and terminating in a rounded tip.

2. The atraumatic dilator of claim 1, wherein the entire filament is pliable.

3. The atraumatic dilator of claim 2, wherein the filament is made of polypropylene.

4. The atraumatic dilator of claim 1, wherein the filament is made of polyurethane.

5. The atraumatic dilator of claim 1, wherein both end portions include a taper and terminate in a rounded tip.

6. The atraumatic dilator of claim 1 wherein the rounded tip is of a material that is relatively softer than the filament.

7. The atraumatic dilator of claim 1, wherein the one end portion of the filament has a greater flexural rigidity than the other end portion of the filament.

8. The atraumatic dilator of claim 7, wherein one end portion of the filament is stainless steel.

9. The atraumatic dilator of claim 7, wherein the end portion of the filament having a lesser flexural rigidity is polypropylene.

10. The atraumatic dilator of claim 1, wherein the taper has an angle with the longitudinal center line of the dilator in the range of about 2 degrees to about 15 degrees.

11. The atraumatic dilator of claim 10, wherein the angle is in the range of about 2 degrees to about 4 degrees.

12. The atraumatic dilator of claim 1 further including at least one penetration depth marker.

13. The atraumatic dilator of claim 1 wherein one end portion thereof has a flexural rigidity in the range of about 1 g·cm² to about 2 g·cm² and the other end portion has a flexural rigidity in the range of about 90 g·cm² to about 100 g·cm².

14. The atraumatic dilator of claim 1 further comprising a stiffener.

15. The atraumatic dilator of claim 14, wherein the stiffener is a stiffening core within the dilator.

16. The atraumatic dilator of claim 15, wherein the stiffening core is a wire.

17. The atraumatic dilator of claim 14, wherein the stiffener is an external sheath disposed about at least a portion of the filament, the external sheath having a rigid distal portion and a flexible proximal portion.

18. The atraumatic dilator of claim 1, wherein the stiffener is a clamp securable about the filament.

19. The atraumatic dilator of claim 1, further provided with tactile signaling element on at least one end portion.

20. The atraumatic dilator of claim 19, wherein the tactile signaling element is a recessed region.

21. The atraumatic dilator of claim 19, wherein the tactile signaling element is a raised region.

21. The atraumatic dilator of claim 1, further provided with a threaded section on the taper.

22. The atraumatic dilator of claim 1, further provided with a retaining device.

23. The atraumatic dilator of claim 22, wherein the retaining device is a collar about the filament.
